(19) United States
(12) Patent Application Publication
(10) Pub. No.: US 2006/0229453 A1
(43) Pub. Date: Oct. 12, 2006

(54) Process for the preparation of 5-[4-[2-[N-methyl-N-(2-pyridyl) amino] ethoxy] phenyl methyl] thiazolidine-2,4-dione maleate (rosiglitazone)

(75) Inventors: Lalji Karsan Gediya, Mumbai (IN); Venkatasubramanian Radha Tarur, Mumbai (IN); Suresh Mahadev Kadam, Navi Mumbai (IN); Subodh Shashikant Patnekar, Thane (IN)

Correspondence Address: PHARMACEUTICAL PATENT ATTORNEYS, LLC, 55 MADISON AVENUE, 4TH FLOOR, MORRISTOWN, NJ, 07960-7397, US

(73) Assignee: USV

(21) Appl. No.: 11/399,789

(22) Filed: Apr. 5, 2006

Related U.S. Application Data
(62) Division of application No. 10/938,317, filed on Sep. 10, 2004.

(30) Foreign Application Priority Data
Jan. 28, 2004 (IN) 80/MUM/2004

(51) Int. Cl. C07D 417/02

(52) U.S. Cl. 546/269.7

(57) ABSTRACT

A process for the preparation of 5-[4-[2-[N-methyl-N-(2-pyridyl)amino]ethoxy]phenyl methyl]thiazolidine-2,4-dione maleate (the compound of Formula VI) comprising the steps of Coupling 2-[N-methyl-N-(2-pyridyl)amino]ethanol (the compound of Formula I) and 4-fluorobenzaldehyde (the compound of Formula II) in N,N-dimethylformamide, isolating the coupled product 4-[2-[N-methyl-N-(2-pyridyl)amino]ethoxy]benzaldehyde (the compound of Formula III), converting said isolated benzaldehyde compound of formula III) to 5-[4-[2-[N-methyl-N-(2-pyridyl)amino]ethoxy]benzylidene]thiazolidine-2,4-dione (the compound of Formula IV) and purifying the same, reducing 5-[4-[2-[N-methyl-N-(2-pyridyl)amino]ethoxy]benzylidene]thiazolidine-2,4-dione, by a novel reduction method for making 5-[4-[2-[N-methyl-N-(2-pyridyl)amino]ethoxy]phenyl methyl]thiazolidine-2,4-dione (the compound of Formula V). This reduction method involves reacting the compound of formula IV with a novel metal legand complex and a reducing agent, purifying the product of formula V obtained by a new method reported in the present invention and converting the said thiazolidine-2,4-dione (the compound of Formula V) into a pharmaceutically acceptable salt.

### RELATED APPLICATIONS

This application claims priority from India National patent Application No. 80/MUM/2004, filed 28 Jan. 2004 and amended 12 Jul. 2004.

### GOVERNMENT INTEREST

None.

The following specification describes the nature of the invention and the manner in which it is to be performed:

### FIELD OF THE INVENTION

The present invention relates to a process for the preparation of 5-[4-[2-[N-methyl-N-(2-pyridyl) amino]ethoxy]phenyl methyl]thiazolidine-2,4-dione], a compound with the structure illustrated below as compound (V) (and sometimes referred to here simply as “(V)”). This compound is known in the art as “rosiglitazone.” It is an antidiabetic compound, the drug of choice for non-insulin-dependent diabetes mellitus (NIDDM). The invention further relates to the novel process of reduction and subsequent purification, which results in substantially pure rosiglitazone and its salts in better yields.

### BACKGROUND AND PRIOR ART

U.S. Pat. No. 5,002,953 discloses the process for reducing 5-[4-[2-[N-methyl-N-(2-pyridyl)amino]ethoxy]benzylidene]thiazolidine-2,4-dione (shown herein as the compound of structure IV) to 5-[4-[2-[N-methyl-N-2-(pyridyl)amino]ethoxy]phenyl methyl]thiazolidine-2,4-dione (V) by using hydrogen on palladium catalyst in 1,4-dioxane. Such process that involves use of noble metal is always costly. Secondly it has inherent problems of safety as noble metal is used. Yield and poisoning of catalyst are other issues, which make it a secondary choice. WO 99,23095 relates to similar process in glacial acetic acid.

Bio organic Medicinal Chemistry Letters, 1994, Vol. 4, 1181-84 discloses the use of magnesium metal and methanol for reduction of 5-[4-[2-[N-methyl-N-(2-pyridyl)amino]ethoxy]benzylidine]thiazolidine-2,4-dione (IV) to 5-[4-[2-[N-Methyl-N-(2-pyridyl)amino]ethoxy]phenyl methyl]thiazolidine-2,4-dione(V). Use of large quantities of magnesium metal, formation of alkoxide with methanol are inherent drawbacks of this process, which necessitate a better option if available. Other associated drawbacks include uncontrolled evolution of hydrogen and therefore safety issues, removal of magnesium alcoholate from methanol, discoloration etc.

WO 93/10254 relates to bio-transformation by Rhodotorula Yeast for conversion of 5-[4-[2-[N-methyl-N-(2-pyridyl)amino]ethoxy]benzylidine]thiazolidine-2,4-dione (IV) to 5-[4-[2-[N-methyl-N-2-(pyridyl)amino]ethoxy]phenyl methyl]thiazolidine-2,4-dione (V). Such biotransformations always involve lot of capital expenditure and process is highly sensitive and therefore prone for failures. Precise controls and sensitivity being the main drawbacks.

WO 98/37073 provides a reduction method using Lithium borohydride/THF/Pyridine, NaBH₄/LiCl/Pyridine and Lithium tri-s-butyl borohydride.

U.S. Pat. No. 5,002,953 and WO 99/23095 disclose reduction of double bond for the preparation of 5-[4-[2-[N-methyl-N-(2-pyridyl)amino]ethoxy]phenyl methyl]thiazolidine-2,4-dione (V). The disclosure has inherent drawbacks. It involves a troublesome step, requires high-pressure hydrogenation using palladium supported on carbon catalyst. In this process high amount of palladium was required which indirectly enhances the cost as well as safety concerns i.e. while handling the catalyst. Also the yield was about 70-80%. In the said process poisoning of catalyst was observed due to thiazolidinedione moiety containing sulphur and hence at times reaction needed longer time for completion.

U.S. Pat. No. 6,632,947 relates to the preparation of 5-[4-[2-[N-methyl-N-(2-pyridyl)amino]ethoxy]benzyl]-2,4-thazolidinedione, by reducing 5-[4-[2-[N-methyl-N-(2-pyridyl)amino]ethoxy]benzylidene]lithium or potassium hydride/lithium tri-sec-butyl borohydride/lithium aluminum hydride in the presence of pyridine.

In case of metal reduction (Reported in Bio. Med. Chem. Lett. 1994, Vol 4, 1181-84) a large quantity of magnesium metal is required, as it forms alkoxide with methanol, aggravating the work up procedure making it more tedious and cumbersome. Further, usage of excess magnesium in methanol causes uncontrolled evolution of hydrogen that can lead to safety hazards. Lastly, the removal of magnesium alcoholate of methanol from the 5-[4-[2-[N-methyl-N-(2-pyridyl)amino]ethoxy]phenyl methyl]thiazolidine-2,4-dione (V) is difficult, yielding to lower isolation efficiency and in turn imparts colour to the final product.

WO 98/37073 discloses biotransformation of a 5-[4-[2-[N-methyl-N-(2-pyridyl)amino ethoxy]benzylidene]thiazolidine-2,4-dione (IV) to its corresponding benzyl derivative was reported by Rhodotorula rubra. However, it is time consuming and difficult to implement on the plant scale, requiring highly sophisticated infrastructure to grow the enzyme.

The following cited references relate to use of cobalt chloride and other salts in combination with borohydride and at times with a ligand for reduction of double bonds preferably in α,βunsaturated compounds, alkenes, alkyl halides etc:

Leutenegger U. Leutengga et. al., Angew. Chem. Int. Ed., 28: 60 (1989) discloses enantioselective reduction of α,β-unsaturated carboxylates with sodium borohydride and catalytic amounts of chiral cobalt semi-corrin complexes. Semi-corrins have appeared to offer ideal prerequisites for the use in enantioselective catalysis with chiral metal complexes. In the presence of catalytic amounts (˜1%) of the semicorrin complex formed in situ from CoCl₂ and the ligand, smooth remarkable uniform reduction to the optically active ester using sodiumborohydride is described. The enantioselectivity have been achieved up to 97%. CoCl₂ and Cobalt bis(semi-corrinate) complex can be recovered in good yield by decomplexation with acetic acid.

M. N. Ricroch and A. Gandemer, J. Organometal. Chem. 67: 119 (1974) discloses (pyridinalo) cobaloxime, chloro(pyridinalo) cobaloxime and Vitamin B₁₂ catalysing, the hydrogenation of α,β-unsaturated esters by hydrogen or sodium borohydride.

J. O. Oshy et al., J. Amer. Chem. Soc. 108:67-72 (1986) discloses transition metal (i.e. cobalt) assisted NaBH₄ and LiAlH₄ reductions for nitrites, alkenes and alkyl halides. The selective reduction of alkenes by NaBH₄—CoCl₂ is reported. It is a typical example of heterogeneous catalytic reduction. Other combinations of LiAlH₄ with CoCl₂ are reported for reduction of alkyl halide. A radical mechanism involving halide atom or oxidative addition to the aluminide is proposed.

Cantello et. al., J. Med. Chem., 1994, 37: 3977-3985 has reported the reduction of 5-[[4-[2-[N-Methyl-N-2-(Pyridyl)amino]ethoxy]benzylidine]thiazolidone-2,4-dione to 5-[[4-[2-[N-Methyl-N-2-(Pyridyl)amino]ethoxy]phenyl methyl]thiazolidone-2,4-dione in magnesium/methanol via electron transfer. The yield reported is 62%.

Pool et al., WO 94/05659, have described the preparation of maleate in ethanol at reflux temperature.

WO 064892, 1999 relates to recrystallization of maleate salt in ethanol/water mixture at 70° C. Further it claims a novel polymorph using the same solvent.

WO 064893 discloses uses of denatured ethanol (5% methanol) for making a novel maleate salt.

WO 064896 describes the preparation of a novel polymorphic maleate salt in acetone under stream of nitrogen for 17.5 hrs at reflux temp.

In all the above reported inventions, pure maleate salt is obtained using mixture of solvents in 75-90% yield.

Various solvents are being industrially used for various processes that are process specific, product specific. Many a times an attempt to use different class of solvents frustrates the purpose. Such classifications are often being given on the basis of polarity, behavioral characteristics, number of polar positions present in the molecule and so on. Another way to represent the same includes use of dielectric constants values of the solvents. The representative chart of solvents with their dielectric constants is listed in Handbook of Chemistry and Physics, by David R. Lide, 81^{st} Edition, Page 6-149 to 6-171 for reference.

The prior art reported above for the reduction of the benzylidene compound (IV) can be said to have the following not so favorable attributes which if overcome would be of immense industrial advantage:
1) U.S. Pat. No. 5,002,953 and WO 99/23095 uses palladium, which is very expensive, unsafe and hazardous.
2) Mg metal in methanol (Reported in Bio. Med. Chem. Lett. 1994, Vol 4, 1181-84) has the inherent problem of difficulty to control the reaction during scale up.
3) Bio transformation requires special infrastructure and
4) WO 98/37073 uses LiBH4, which is extremely expensive

### OBJECTS OF INVENTION

Our invention provides a novel and an industrially viable and cost-effective process for the preparation of rosiglitazone maleate, which obviates the drawbacks of prior-art process by use of cheaper and easily available raw-materials.

Our invention provides 5-[4-[2-[N-methyl-N-(2-pyridyl)amino]ethoxy]phenyl methyl]thiazolidene-2,4-dione (the compound of Formula V) in high yield and purity, by the reduction of 5-[4-[2-[N-methyl-N-2-(pyridyl)amino]ethoxy]benzylidene]thiazolidine-2,4-dione (the compound of Formula IV).

Our invention also provides a purification method for purifying the intermediate compound of Formula (IV) to the compound of Formula (V), to achieve high purity.

Using our invention, one may obtain the pharmaceutically-acceptable salt, viz. rosiglitazone maleate from rosiglitazone base in high yield and purity, by using an acetone\isopropyl alcohol (IPA) mixture.

### SUMMARY OF THE INVENTION

The present invention provides a process for reducing 5-[4-[2-[N-methyl-N-(2-pyridyl)amino]ethoxy]benzylidine]thiazolidine-2,4 dione (the compound of Formula IV) to 5-[4-[2-[N-methyl-N-(2-pyridyl)amino]ethoxy]phenyl methyl]thiazolidine 2,4-dione (the compound of Formula V) using cobalt ion, a ligand and a reducing agent. This process employs temperature in the range of 20-45° C. and wherein a suitable solvent which is mixture of solvents is used, viz. tetrahydrofuran (THF)/dimethyl formamide (DMF)/Water.

The novel purification route selected gives substantially pure product. Conventionally inorganic metal when loosely bonded to organic substrate, the adduct is called complex. In the present invention ammonia forms a loose adduct with organic substrate, which is referred as complex and be construed accordingly.

### BRIEF DESCRIPTION OF THE DRAWINGS

FIG. 1 is a diagram of the rosiglitazone maleate synthetic scheme

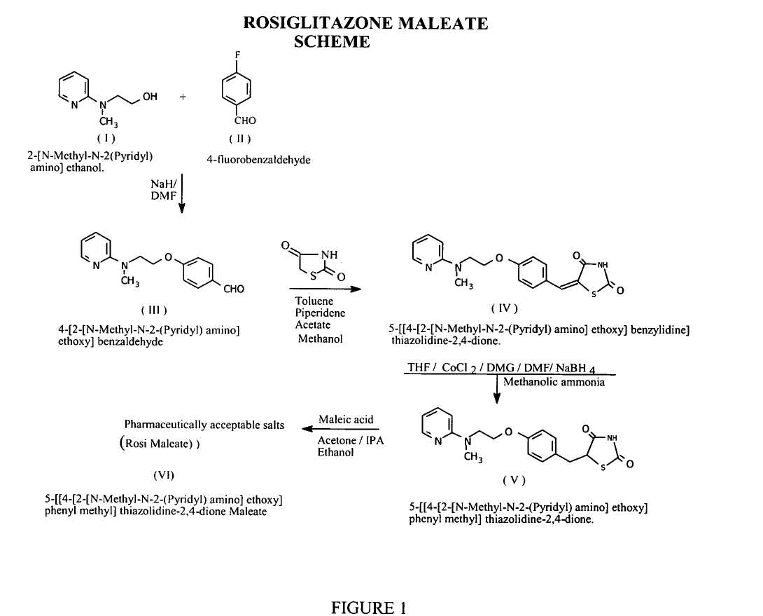

### DETAILED DESCRIPTION OF THE INVENTION

The present invention offers a novel reduction method, which is more efficient because it is faster, easier and results in substantially improved yield of the desired product. It is also more convenient for scale up at plant, since no high-pressure autoclaves are required. The solvents used for this process are THF, DMF and water by alone or a mixture thereof.

Various solvents with different constitution are used which are process specific, product specific. Hydroxylic solvents are to be construed to mean solvents whose molecular formula has hydroxyl group as electronegative part of molecule The process of reduction may be facilitated by the use of any of a diverse group of solvents, including solvents with heteroatoms present in the molecular formula. The use of a ligand and complexing agent employed for our process is adequately elaborated in following examples, e.g., Example No. 2

Use of borohydride of alkali metal in the reduction process gives an impressive yield. The yield is high—in the range of 90-95%, yielding product with a purity of about 97%, as measured by HPLC.

Alcoholic purification of the intermediate compound of Formula (IV) enhances purity of the resulting compound of Formula (V) to 97 to 97.5%, as measured by HPLC, where as the crude product has purity in the range of 88-90%.

Purification of 5-[4-[2-[N-methyl-N-(2-pyridyl)amino]ethoxy]phenyl methyl]thiazolidine-2,4-dione (the compound of Formula V) was accomplished by dissolving it in alcohol at alkaline pH obtained by purging dry ammonia gas. This on subsequent neutralization with acetic acid yielded pure compound purity 99% by HPLC.

Further preparation of maleate from purified base (the compound of Formula V) provides the final product i.e. 5-[4-[2-[N-methyl-N-(2-pyridyl)amino]ethoxy]phenyl methyl]thiazolidine-2,4-dione maleate (the compound of Formula VI) with impurity level below 0.1%.

The present invention discloses a process for the preparation of 5-[4-[2-[N-methyl-N-(2-pyridyl) amino]ethoxy]phenyl methyl]thiazolidine-2,4-dione maleate (the compound of Formula VI) comprising the steps of:
1) Coupling 2-[N-methyl-N-(2-pyridyl)amino]ethanol (the compound of Formula I) and 4-fluorobenzaldehyde (the compound of Formula II) in N,N-dimethylformamide with sodium hydride as a base in a known manner.
2) Isolating the coupled product 4-[2-[N-methyl-N-(2-pyridyl)amino]ethoxy]benzaldehyde (the compound of Formula III).
3) Converting said isolated benzaldehyde compound (the compound of Formula III) to 5-[4-[2-[N-methyl-N-(2-pyridyl)amino]ethoxy]benzylidene]thiazolidine-2,4-dione (the compound of Formula IV) in known manner.
4) Purifying the coupled product (the compound of Formula IV) with hydroxylic solvents like lower carbon chain alcohols preferably C₁-C₄ aliphatic alcohols including straight and branched chain alcohols.
5) Reduction of 5-[4-[2-[N-methyl-N-(2-pyridyl)amino]ethoxy]benzylidene]thiazolidine-2,4-dione (IV) to 5-[4-[2-[N-methyl-N-(2-pyridyl)amino]ethoxy]phenylmethyl]thiazolidine-2,4-dione (the compound of Formula V) by a novel reduction method. This reduction method involves reacting the compound of Formula IV with metal ligand complex and a reducing agent in hydroxylic solvents at a controlled temperature ranging from 10-50° C. under alkaline condition, pH in the range of 9 to 11. Metal ion of metal ligand complex is selected from bivalent metals, preferably cobalt in the form of cobalt chloride and cobalt diacetate. The said ligand of metal ligand complex is an aromatic or aliphatic ligand, preferably bidentate selected from dimethyl glyoxime and 2,2′-bipyridyl. Reducing agents used may be a hydride of group III metal with an alkali metal, such as, for example, sodium borohydride, potassium borohydride or lithium borohydride. Optionally, lithium aluminum hydride is also used in the said reduction process. Suitable temperature conditions for the reduction is 10-50° C. Preferable temperature condition for the said reduction reaction is 20-40° C. Preferred temperature condition for the reduction reaction is 25 to 35° C. Solvent for the said reduction reaction is preferably selected from methanol, ethanol, isopropyl alcohol, DMF, THF in combination with other solvents like methanol, ethanol or IPA in combination with water.
6) Purifying the product (V) obtained by a novel method as described in the present invention, which comprises of treatment with complexing agent in alcohol under basic complexing conditions. Alcohols used in the said reaction is lower branched or unbranched aliphatic alcohols like ethanol, methanol, isopropyl alcohol or tert-butanol either alone or mixture thereof with basic complexing conditions. Basic complexing agent used here is non-aqueous gaseous ammonia, which is purged into alcohol under controlled condition. Optionally, non-aqueous liquefied ammonia is also used. The optimum basic condition is pH in the range of 8-12, preferred 9-10.
7) Neutralization of the reaction mixture obtained from step 6 with acid before formation of maleate with weak organic or inorganic acid in diluted form, preferably acetic acid.
8) Converting the thiazolidine-2,4-dione compound of Formula V into a pharmaceutically acceptable salt by treating it with maleic acid in a mixture of solvents like acetone and isopropyl alcohol under controlled temperature. The ratio of acetone to isopropyl alcohol varies from about 5:95 to about 95:5. The temperature range for salt formation is about 20-40° C., preferably 25-30° C.

### EXPERIMENTAL FINDINGS

### Example 1

### Purification of compound 5-[4-[2-[N-methyl-N-(2-pyridyl)amino]ethoxy]benzylidene]thiazolidine-2,4-dione (the compound of Formula IV)

To a 100 ml 3-necked round bottom flask, equipped with a mechanical stirrer is charged 10 gms of compound of formula 5-[4-[2-[N-methyl-N-(2-pyridyl)amino]ethoxy]benzylidene]thiazolidine-2,4-dione (IV). To this 25 ml of methanol is added. The whole solution is refluxed for 1 hour. The reaction mass is then cooled at 10° C., stirred for 1 hour, filtered, washed with 25 ml of cold methanol and dried at 70° C. for 6 hrs. Yield of product (IV) is 8 gms. Purity is 97% by HPLC.

### Example 2

### Preparation of compound 5-[4-[2-[N-methyl-N-(2-pyridyl)amino]ethoxy]phenyl methyl]thiazolidine-2,4-dione (V)

To a 500 ml 3-necked round bottom flask, equipped with a mechanical stirrer is charged. 10 gms of compound (IV), 140 ml of water, 34 ml of tetrahydrofuran and 12 ml of 1.0 N sodium hydroxide. The mixture is stirred at 25° C. for 10 min and cooled to 15° C. To the cooled mixture is added 30 ml of catalyst solution, prepared by dissolving 1.3 gms of dimethylglyoxime and 0.068 gm of cobaltous chloride hexahydrate in 28 ml of dimethylformamide. Then a solution containing 2.13 gms of sodium borohydride in 2.8 ml of 1N aqueous sodium hydroxide and 20 ml of water is added to the reaction mixture at the rate of 01 ml/min. The reaction mass is then stirred at 15° C. for 4 hours. The reaction is neutralised with 8-10 ml of acetic acid. Solid precipitated out is quenched in 50 ml of water. Solid product is filtered, washed with 50 ml of water and dried. Yield is 9.2 gms (91.5%). Purity by HPLC is 97.5%.

### Example 3

### Purification of 5-[4-[2-[N-methyl-N-(2-pyridyl)amino]ethoxy]phenylmethyl]thiazolidine-2,4-dione (V) by alcoholic ammonia

To a 100 ml 3-necked round bottom flask, equipped with a mechanical stirrer is charged 10 gms of compound 5-[4-[2-[N-methyl-N-(2-pyridyl)amino]ethoxy]phenyl methyl]thiazolidine-2,4-dione (V). The compound is taken in a 50 ml methanol and 20 ml of ethanol, cooled it to 10-15° C. Dry ammonia gas is purged in the reaction mixture till the solution became clear which is further stirred for 10-15 min. 5% charcoal is added, stirred for half an hour and filtered through hy-flow bed. The reaction mixture is cooled to 10-15° C. Acetic acid is added drop wise within 30-35 min maintaining temperature 10-15° C. The solid product is precipitated at pH 6-6.6 which is filtered and washed with 25 ml of cold methanol (10° C.). The product is filtered and dried at 65° C. for 6 hrs. Yield is 9 gms (90%). Purity by HPLC is 99%.

### Example 4

### Preparation of 5-[4-[2-[N-methyl-N-(2-pyridyl)amino]ethoxy]phenyl methyl]thiazolidine-2,4-dione maleate (VI)

To a 250 ml 3-necked round bottom flask, equipped with a mechanical stirrer is charged 10 gms of 5-[4-[2-[N-methyl-N-(2-pyridyl)amino]ethoxy]phenyl methyl]thiazolidine-2,4-dione (V) and 3.5 gms of maleic acid. Slowly 80 ml of acetone is added in the mixture. Further 80 ml of IPA is added to the solution. Separated solid is filtered, washed with 40-50 ml of IPA and dried. Yield is 10.5 gms (80%). Purity by HPLC is 99.5%.

While the present invention is described above in connection with preferred or illustrative embodiments, these embodiments are not intended to be exhaustive or limiting of the invention. Rather, the invention is intended to cover all alternatives, modifications and equivalents included within its scope, as defined by the appended claims.

We use the singular to include one, as well as more than one. For example, the claim term “a compound selected from A, B and C” covers one of the three enumerated compounds, and two together, and even all three together.

We claim:

1. A process comprising:
a. reducing 5-[4-[2-[N-methyl-N-(2-pyridyl)amino]ethoxy]benzylidene]thiazolidine-2,4-dione with:
i. hydroxylic solvent, and
ii. metal-ligand complex, and
iii. reducing agent;
to form 5-[4-[2-[N-methyl-N-(2-pyridyl)amino]ethoxy]phenyl methyl]thiazolidine-2,4-dione;
b. combining said 5-[4-[2-[N-methyl-N-(2-pyridyl)amino]ethoxy]phenyl methyl]thiazolidine-2,4-dione with complexing agent under basic pH complexing condition to create a basic pH complexing mixture;
c. neutralizing said basic pH complexing mixture; and
d. adding to said 5-[4-[2-[N-methyl-N-(2-pyridyl)amino]ethoxy]phenyl methyl]thiazolidine-2,4-dione, maleic acid in an amount sufficient to create 5-[4-[2-[N-methyl-N-(2-pyridyl)amino]ethoxy]phenyl methyl]thiazolidine-2,4-dione maleate.

2. The invention of claim 1, wherein the said metal-ligand complex comprises a bivalent metal.

3. The invention of claim 2, wherein said bivalent metal comprises cobalt.

4. The invention of claim 3, wherein said cobalt is present in a form selected from the group consisting of: cobalt chloride and cobalt diacetate.

5. The invention of claim 1, wherein said metal-ligand complex comprises bidentate ligand.

6. The invention of claim 5, wherein said bidentate ligand is aromatic or aliphatic.

7. The invention of claim 6, wherein said bidentate ligand is selected from the group consisting of: 2,2′-bipyridyl and dimethyl glyoxime.

8. The invention of claim 1, wherein said reducing agent is selected from the group consisting of: hydride of a Group III metal, and alkali metal.

9. The invention of claim 8, wherein said reducing agent is selected from the group consisting of: hydride of boron, sodium borohydride, potassium borohydride and lithium borohydride.

10. The invention of claim 9, wherein said metal-ligand complex comprises cobaltous chloride and dimethyl glyoxime, and said reducing agent comprises sodium borohydride.

11. The invention of claim 10, said reducing performed at a temperature of from about 10 to about 50° C., and at a pH of from about 9 to about 11.

12. The invention of claim 11, wherein said temperature range is from about 25 to about 35° C.

13. The invention of claim 1, wherein said hydroxylic solvent is selected from the group consisting of: methanol, ethanol, isopropyl alcohol, dimethylformamide, tetrahydrofuran, and water.

14. The invention of claim 1, wherein said basic pH complexing mixture includes alcohol.

15. The invention of claim 14, wherein said alcohol is a lower carbon chain aliphatic alcohol.

16. The invention of claim 1, wherein the said complexing agent comprises non-aqueous ammonia.

17. The invention of claim 1, wherein the said basic pH complexing condition is a pH from about 9 to about 10.

18. The invention of claim 1, wherein said neutralizing the pH of said basic pH complexing mixture comprises adding acetic acid.

19. The invention of claim 1, said adding maleic acid step performed in organic solvent and alcohol.

20. The invention of claim 19, said organic solvent comprising acetone, said alcohol comprising isopropyl alcohol, and said adding maleic acid is carried out at a temperature from about 20 to about 40° C.

21. A process comprising:
a. reducing 5-[4-[2-[N-methyl-N-(2-pyridyl)amino]ethoxy]benzylidene]thiazolidine-2,4-dione under alkaline condition with:
i. hydroxylic solvent comprising dimethylformamide, tetrahydrofuran and water, and
ii. cobalt-dimethyl glyoxime complex, and
iii. sodium borohydride;
to form 5-[4-[2-[N-methyl-N-(2-pyridyl)amino]ethoxy]phenyl methyl]thiazolidine-2,4-dione;
b. combining said 5-[4-[2-[N-methyl-N-(2-pyridyl)amino]ethoxy]phenyl methyl]thiazolidine-2,4-dione with complexing agent under basic pH complexing condition to create a basic pH complexing mixture;
c. neutralizing the pH of said basic pH complexing mixture; and
d. adding to said 5-[4-[2-[N-methyl-N-(2-pyridyl)amino]ethoxy]phenyl methyl]thiazolidine-2,4-dione, maleic acid in an amount sufficient to create 5-[4-[2-[N-methyl-N-(2-pyridyl)amino]ethoxy]phenyl methyl]thiazolidine-2,4-dione maleate.

22. The invention of claim 21, wherein said dimethylformamide and tetrahydrofuran present in relative proportions of from about 1:2 to about 1:1.

23. A process comprising: combining 5-[4-[2-[N-methyl-N-(2-pyridyl)amino]ethoxy]benzylidene]thiazolidine-2,4-dione with metal-ligand complex; and forming 5-[4-[2-[N-methyl-N-(2-pyridyl)amino]ethoxy]phenyl methyl]thiazolidine-2,4-dione.

24. The invention of claim 23, wherein the said metal-ligand complex comprises a bivalent metal.

25. The invention of claim 24, wherein said bivalent metal comprises cobalt.

26. The invention of claim 25, wherein said cobalt is present in a form selected from the group consisting of: cobalt chloride and cobalt diacetate.

27. The invention of claim 23, wherein said 5-[4-[2-[N-methyl-N-(2-pyridyl) amino]ethoxy]phenyl methyl]thiazolidine-2,4-dione comprises 5-[4-[2-[N-methyl-N-(2-pyridyl) amino]ethoxy]phenyl methyl]thiazolidine-2,4-dione maleate.
